(19) United States
(12) Patent Application Publication
(10) Pub. No.: US 2013/0237570 A1
(43) Pub. Date: Sep. 12, 2013

(54) Formulation and Use and Manufacture Thereof

(71) Applicant: MCNEIL AB, Helsingborg (SE)

(72) Inventors: Katarina E.A. Lindell, Eslov (SE); Anette K. Schluter, Rydeback (SE); Gunner A. Bergengren, Bjuv (SE); Bengt A. Bosson, Helsingborg (SE)

(73) Assignee: McNeil AB, Helsingborg (SE)

(21) Appl. No.: 13/871,476

(22) Filed: Apr. 26, 2013

Related U.S. Application Data
(63) Continuation of application No. 12/619,851, filed on Nov. 17, 2009.
Application No. 12/619,851 is a continuation of application No. 11/686,842, filed on Mar. 15, 2007.
Application No. 11/686,842 is a division of application No. 10/345,676, filed on Jan. 16, 2003, now abandoned.
(60) Provisional application No. 60/351,178, filed on Jan. 21, 2002.

(51) Int. Cl. A61K 9/00; A61K 45/06; A61K 31/465

(52) U.S. Cl. CPC A61K 9/0002; A61K 31/465; A61K 45/06
USPC 514/343

(57) ABSTRACT

This invention relates to a liquid pharmaceutical formulation for delivering nicotine to a subject. This invention also relates to a method and a system for delivering nicotine as well as manufacturing and use of said liquid pharmaceutical formulation.

### CROSS REFERENCE TO RELATED PATENT APPLICATIONS

The present application is a continuation of pending U.S. patent application Ser. No. 12/619,851, filed Nov. 17, 2009, which is a continuation of pending U.S. patent application Ser. No. 11/686,842, filed Mar. 15, 2007, which is a divisional of U.S. patent application Ser. No. 10/345,676 filed Jan. 16, 2003, now abandoned, which claims the benefit of U.S. Provisional Patent Application Ser. No. 60/351,178, filed Jan. 21, 2002. All of the foregoing applications are incorporated herein by reference in their entireties for any and all purposes.

### FIELD OF THE INVENTION

This invention relates to a liquid pharmaceutical formulation for delivering nicotine to a subject. This invention also relates to a method and a system for delivering nicotine as well as manufacturing and use of said liquid pharmaceutical formulation.

### BACKGROUND OF THE INVENTION

### Tobacco Dependence and Reduction Thereof

In recent years, with the recognition of the harmful effects of tobacco smoking, there have been numerous campaigns and programs by governmental agencies and various health groups and other interested organisations to disseminate information about the adverse health effects resulting from tobacco smoking. Moreover, and as a result of this recognition of the harmful effects, there have been many programs directed to attempts in reducing smoking incidence.

Nicotine is an organic compound and is the principal alkaloid of tobacco. Nicotine is the chief active ingredient in the tobacco used in cigarettes, cigars, snuff and the like. Nicotine is also an addictive drug, though, and smokers characteristically display a strong tendency to relapse after having successfully stopped smoking for a time. Nicotine is the world's second most used drug, after caffeine from coffee and tea.

The main problem with tobacco smoking is its enormous implications on health. Today it is estimated that smoking-related diseases cause some 3-4 million deaths per year. In the US Surgeon General's 1988 report on The Health Consequences of Smoking, it was estimated that in the US alone about 300,000 deaths are caused each year by diseases related to cigarette smoking. In fact, excessive smoking is now recognized as one of the major health problems throughout the world. This grim consequence of tobacco smoking has urged many medical associations and health authorities to take very strong actions against the use of tobacco.

Even though tobacco smoking is decreasing in many developed countries today it is hard to see how the societies could get rid of the world's second most used drug.

The most advantageous thing a heavy smoker can do is to reduce or preferably even stop smoking completely. Experience shows, however, that most smokers find this extremely difficult since, mostly, tobacco smoking result in a dependence disorder or craving. The WHO has in its International Classification of Disorders a diagnosis called Tobacco Dependence. Others, like the American Psychiatric Association call the addiction Nicotine Dependence. It is generally accepted that these difficulties to stop smoking result from the fact that those heavy smokers are dependent on nicotine. The most important risk factors are, however, substances that are formed during the combustion of tobacco, such as carcinogenic tar products, carbon monoxide, aldehydes, and hydrocyanic acid.

### Effects of Nicotine

The administration of nicotine can give satisfaction and the usual method is by smoking, either by smoking e.g., a cigarette, a cigar or a pipe, or by snuffing or chewing tobacco. However, smoking has health hazards and it is therefore desirable to formulate an alternative manner of administering nicotine in a pleasurable manner that can be used to facilitate withdrawal from smoking and/or used as a replacement for smoking.

Upon smoking of a cigarette, nicotine is quickly absorbed into the smoker's blood and reaches the brain within around ten seconds after inhalation. The quick uptake of nicotine gives the consumer a rapid satisfaction, or kick. The satisfaction, then, lasts during the time of smoking the cigarette and for a period of time thereafter. The poisonous, toxic, carcinogenic, and addictive nature of smoking has provided efforts for methods, compositions and devices, which help in breaking the habit of smoking.

Nicotine is an addictive poisonous alkaloid C5H4NC4H7NCH3, derived from the tobacco plant. Nicotine is also used as an insecticide. Approximately forty milligrams of nicotine may kill an adult (Merck Index).

### Nicotine Replacement Products

One way to reduce smoking is to provide nicotine in a form or manner other than by smoking and some products have been developed to fulfill this need. Nicotine containing formulations are currently the dominating treatments for tobacco dependence.

The success in achieving reduction in the incidence of smoking has been relatively poor using presently known products. State of the art involves both behavioral approaches and pharmacological approaches. More than 80% of the tobacco smokers who initially quit smoking after using some behavioral or pharmacological approach to singly reduce smoking incidence generally relapse and return to the habit of smoking at their former rate of smoking within about a one year's period of time.

As an aid for those who are willing to stop smoking there are several ways and forms of nicotine replacement products available on the market, such as nicotine chewing gums according to U.S. Pat. No. 3,845,217. Several methods and means have been described for diminishing the desire of a subject to use tobacco, which comprises the step of administering to the subject nicotine or a derivative thereof as described in e.g., U.S. Pat. No. 5,939,100 (nicotine containing microspheres) and U.S. Pat. No. 4,967,773 (nicotine containing lozenge).

The effects of pH on the absorption of nicotine is discussed e.g., in Eur J Clin Pharmacol, Vol. 56, 2001, pages 813-818, L. Molander et al, “Pharmacokinetic investigation of a nicotine sublingual tablet”. The effects of pH on a liquid nicotine formulation for administration to the oral cavity are though not disclosed.

The use of skin patches for transdermal administration of nicotine has been reported (Rose, in Pharmacological Treatment of Tobacco Dependence, (1986) pp. 158-166, Harvard Univ. Press). Nicotine-containing skin patches that are in wide use today can cause local irritation and the absorption of nicotine is slow and affected by cutaneous blood flow.

Nicotine-containing nose drops have been reported (Russell et al., British Medical Journal, Vol. 286, p. 683 (1983); Jarvis et al., British Journal of Addiction, Vol. 82, p. 983 (1987)). Nose drops, however, are difficult to administer and are not convenient for use at work or in other public situations. Ways of administrating nicotine by way of delivery directly into the nasal cavity by spraying is known from U.S. Pat. No. 4,579,858, German Application No. DE 32 41 437 and International Publication No. WO/93 127 64. There may, though, be local nasal irritation with use of nasal nicotine formulations. The difficulty in administration also results in unpredictability of the dose of nicotine administered.

Also, inhaling devices resembling a cigarette are known for uptake of nicotine vapors as suggested in U.S. Pat. No. 5,167,242. An aerosol for deposing nicotine in the lungs is disclosed in German Application No. DE 32 41 437.

Mouth sprays comprising nicotine are known in the art, e.g., according to U.S. Pat. No. 6,024,097 wherein is disclosed a method of assisting a smoker in giving up the smoking habit whereby is used a plurality of aerosol dispensers comprising progressively lesser concentrations of nicotine. The aerosol is intended to be administered into the mouth. The liquid in the dispensers essentially consists of nicotine and alcohol.

A similar mouth spray is disclosed in U.S. Pat. No. 5,810,018, whereby in addition the aerosol comprises progressively greater concentrations of at least one selected stimulant.

International Publication No. WO 98/24420 discloses an aerosol device with an active and a propellant. The device may be used for e.g., sublingual administration. Nicotine is mentioned as an active in a long “laundry list” of drugs. There are though no examples on nicotine formulations.

U.S. Pat. No. 5,721,257 discloses a method for treating a condition responsive to nicotine therapy comprising a first treatment with transdermally administered nicotine and a second treatment with transmucosally administered nicotine. It is stated that the transmucousal administration may be accomplished via an aerosol to the nasal membranes. No administration to the oral cavity is disclosed.

International Publication No. WO 97/38663 discloses a buccal aerosol spray using a non-polar solvent. Nicotine is mentioned as one useful active in this spray.

U.S. Pat. No. 5,955,098 likewise discloses a buccal non-polar spray wherein nicotine may be an active.

None of the known mouth sprays comprise any buffering and/or pH regulating means.

### Prior Art and Problems Thereof

The captioned means and methods do not satisfy the craving that certain users of tobacco experience. Specifically these means and methods generally do not provide for a sufficiently rapid uptake of nicotine without adverse effects.

This means that none of the hitherto known means and methods satisfactorily fulfills the following well-known NRT teaching by Russel et al:

I: A fast delivery or “boost” of nicotine, sufficiently rapid to give positive subjective nicotine effects in contrast with current nicotine gums and patches, will lead to faster craving relief, and

II: faster craving relief will give better craving control, and

III: better craving control should result in higher overall quit rates.

For the captioned see Russel, M. A. H., Stapleton, J. A. and Feyerabend C. Nicotine boost per cigarette as the controlling factor of intake regulation of smokers; In: Clark et al. (Eds.) Effects of Nicotine on Biological Systems II, Advances in Pharmacological Sciences, Birkhäuser Verlag, Basel, (1995) 233-238.

In light of the aforementioned problems there is a strong need and interest to develop means and methods for the administration of nicotine to provide a fast satisfaction to a person craving for nicotine or to provide a sense of smoking satisfaction without smoking, whereby also may be avoided problems associated with the prior art means and methods. The present invention addresses said need and interest.

### SUMMARY OF THE INVENTION

In view of the foregoing disadvantages known in the art when trying to deliver nicotine to a subject so as to obtain a rapid transmucosal uptake of nicotine in the oral cavity of the subject the present invention provides a new and improved product, systems and methods for obtaining a rapid transmucosal uptake of nicotine in the oral cavity of the subject.

Objects of the present invention are to provide an efficient and effective product, as well as methods and systems for a rapid uptake of nicotine in a subject to avoid the disadvantages of previously known products and methods. The present invention also satisfactorily satisfies the above teaching of Russel et al.

Thus, the present invention provides a method for delivering nicotine in any form to a subject comprising administering to a subject a liquid pharmaceutical formulation containing nicotine in any form into the oral cavity of the subject and allowing the nicotine in any form to be absorbed into the systemic circulation of the subject essentially by rapid buccal uptake of nicotine as well as a method for manufacturing said liquid pharmaceutical formulation.

The present invention also provides a method for obtaining reduction of the urge to smoke or use tobacco containing material and/or for providing a fast sense of smoking satisfaction without smoking, comprising the steps of replacing at least partly the tobacco containing material with said liquid pharmaceutical formulation, administering to a subject a liquid pharmaceutical formulation containing nicotine in any form into the oral cavity of the subject and allowing the nicotine to be systemically absorbed by the subject essentially by buccal uptake of nicotine.

Furthermore, the present invention provides a system for delivering nicotine in any form to a subject, comprising said liquid pharmaceutical formulation and at least one other means for obtaining reduction of the urge to smoke or use of tobacco as well as a system for obtaining reduction of the urge to smoke or otherwise use of tobacco and/or for providing a sense of smoking satisfaction without smoking, comprising a liquid pharmaceutical formulation as per above and at least one other method for obtaining reduction of the urge to smoke or otherwise use tobacco. Said system may be a system wherein the at least one other method is selected from the group consisting of administration through chewing gums, nasal sprays, transdermal patches, inhaling devices, lozenges, tablets and parenteral methods, subcutaneous methods, intravenous methods, rectal methods, vaginal methods and transmucousal methods; or other use of tobacco.

The present invention provides for a flexible, convenient and discrete use in comparison with other means for transmucosal delivery of nicotine, e.g., chewing gums, lozenges and tablets. No chewing or sucking is necessary. Further and in contrast to other transmucosal dosage forms the present liquid pharmaceutical formulation provides nicotine in a form being directly buccally absorbable by a subject. Known formulations for nasal delivery of nicotine are inconvenient—side effects include running nose, nasal irritation and irritation of the eyes. The nicotine in chewing gums, lozenges and tablets need pass a transformation phase, involving e.g., mastication, disintegration, melting and/or dissolution, prior to being present in a directly absorbable form. A nicotine patch provides for a discrete administration, but does not provide for a fast uptake of nicotine.

A product according to the present invention is alkalized by buffering and/or pH regulation in such a way that upon administration of the liquid pharmaceutical formulation the pH of the liquid of the oral cavity is increased by 0.3-4 pH units, or preferably increased by 0.5-2.5 pH units.

Use of said product will according to the invention rapidly deliver nicotine in any form to a subject and will also provide for obtaining a quick and/or sustained and/or complete reduction of the urge to smoke or use tobacco and/or for providing a sense of smoking satisfaction without smoking resembling the sense of smoking satisfaction obtained after regular smoking or use of tobacco.

### BRIEF DESCRIPTION OF THE DRAWINGS

FIG. 1 is a diagram showing venous blood plasma level concentrations of nicotine after two different ways of administering nicotine. For both ways of administration one unit dose was administered at time zero. No further doses were administered. 50 persons, all being nicotine users, took part in this test. “Spray” represents 200 μl of a liquid pharmaceutical formulation according to below Example 4 being sprayed under the tongue. This unit dose comprised 3.5 mg nicotine measured as free base. “Microtab” represents one tablet of Nicorette® Microtab, comprising 4 mg nicotine measured as free base. Nicorette® Microtab is pharmacologically equivalent to Nicorette Gum. “Spray” comprises a buffer. “Microtab” comprises no buffer. With “Spray” the liquid pharmaceutical formulation was held in the mouth for one minute before swallowing. With “Microtab” the tablet was kept under the tongue until dissolved. Each symbol on the respective graph represents one measurement of nicotine in venous blood plasma.

FIG. 2 shows mean plasma concentrations after sublingual administration of three liquid pharmaceutical formulations with pH 6, 7 and 8.5 respectively. For each formulation 200 μl was sprayed under the tongue at time zero. For all said three formulations the concentration of nicotine was 10 mg/ml, i.e., each 200 μl spray dose as above contained 2 mg nicotine calculated as free base. The formulation with pH 8.5 was a formulation according to below Example 1. The formulations with pH 7 and pH 6 were formulations according to below Example 2 and Example 3 respectively.

FIG. 3 compares the venous blood nicotine plasma profile vs. time of a single dose of the current Nicorette® Gum, extra strength (4 mg), with the corresponding plasma profile when smoking a “light” (low-nicotine) cigarette. One objective with the present invention is to obtain a buccal nicotine formulation providing for a pK profile being closer to the pK profile for a cigarette than is provided using presently known buccal nicotine formulations.

FIG. 4 shows the mean score values from 52 smoking volunteers in a randomized open study of the “urge to smoke” (craving), as estimated and recorded on a visual analogue scale (VAS) as a function of time when the same formulations as in FIG. 1 were used. The craving scores were recorded directly after smoking one cigarette and during the abstinence of 7 hours before the administration of the nicotine products. The scores were then recorded more frequently during 1 hour after the administration. The heart rate was also monitored in this study. This figure clearly shows that the present invention provides for a much faster reduction of the urge to smoke score than do present buccal nicotine formulations. For example, about 2 minutes after administration of a formulation according to the present invention the craving score is reduced by 50%. With Nicorette® Microtab a 50% decrease in craving score is obtained only more than 10 minutes after administration.

### DETAILED DESCRIPTION OF ILLUSTRATIVE EMBODIMENTS

### Definitions

The terms “tobacco”, “tobacco containing material” and similar are herein intended to mean such material for any type of use of tobacco including smoking, snuffing or chewing whereby is used inter alia a cigarette, a cigar, pipe tobacco, snuff and chewing tobacco.

The term “fast reduction of the urge to smoke or use tobacco ” is herein intended to mean an initial priming of the subject so as to achieve a reduction of the urge to smoke or use tobacco.

The term “transient” is intended to pertain to a non-permanent change of a biological and/or physiological state, upon which after a certain period of time said state will return to its value or behavior prior to said change.

The term “buccal” and “buccally” are herein intended to pertain to all of or any part of the soft tissue lining of the oral cavity.

The term “liquid of the oral cavity” is herein intended to mean saliva and/or saliva mixed with a quantity of the liquid pharmaceutical formulation.

The term “incidence of administration” is herein intended to mean administration of one or more single doses of the liquid pharmaceutical formulation within the same time frame, said time frame being dependent on the needs of the subject receiving the administration, said time frame extending from a few seconds to around ten minutes.

### The Buffering Agent and the pH Regulating Means

Absorption of nicotine from the oral cavity to the systemic circulation is dependent on the pH of the saliva and the pKa of nicotine, which is about 7.8. Assuming a pH of the saliva of 6.8 only about 10% of the nicotine in saliva will be in the free base form. Thus, in order to promote absorption of nicotine in a free base form, which is the form predominantly absorbed through the mucosa, the pH of the saliva must be increased. At a pH of 8.8 about 90% of the nicotine in saliva will then be in the free base form.

Hence and according to the invention, the liquid pharmaceutical formulation is alkalized by buffering and/or pH regulation. This may be achieved by including physiologically acceptable buffering substances or agents, or by other means. With other means it is intended to include buffering by any component in the product, which may not normally act as a buffering agent, such as a self-buffering additive and/or pH regulating forms of nicotine.

By buffering and/or pH regulation thereby increasing the pH of the saliva the uptake of nicotine is changed, e.g., increased compared to the nicotine uptake when the saliva is not alkalized by buffering and/or pH regulation. Also, since the transmucosal uptake of nicotine in the oral cavity according to the invention is faster than for nicotine not being buffered and/or pH regulated according to the invention, less nicotine will be swallowed and reach the gastrointestinal (GI) tract. The nicotine that reaches the GI tract will be subjected to first pass metabolism, which reduces the total amount of intact nicotine absorbed additionally reducing the rate of nicotine absorption. This means that the absorption kinetics of nicotine that is not co-administered with a buffer according to the invention will generally be slower and the bioavailability will generally be lower than when administered together with a buffer.

For buffering may be used one or more buffering agents selected from the group consisting of carbonates including bicarbonate or sesquicarbonate, glycinate, phosphate, glycerophosphate or citrate of an alkali metal, such as potassium or sodium, or ammonium, and mixtures thereof.

Further embodiments may use trisodium or tripotassium citrate, and mixtures thereof.

Still further embodiments may comprise different phosphate systems, such as trisodium phosphate, disodium hydrogen phosphate; and tripotassium phosphate, dipotassium hydrogen phosphate, and calcium hydroxide, sodium glycinate; and mixtures thereof.

Alkali metal carbonates, glycinates and phosphates are preferred buffering agents.

The pH regulation may also be obtained by using pH-regulating forms of nicotine, e.g., nicotine free base.

The amount of the buffering agent or agents in the liquid pharmaceutical formulation is preferably sufficient in the specific embodiments to raise the pH of the saliva to above 7, as specified above and, to maintain the pH of the saliva in the oral cavity above 7, e.g., pH 7-11. Otherwise expressed the liquid pharmaceutical formulation should be alkalized by buffering and/or pH regulation in such a way that upon administration to a subject the pH of the liquid of the oral cavity of the subject is transiently increased by about 0.3-4 pH units, preferably by about 0.5-2.5 pH units. The amount of buffering agent(s) required to achieve such an increase in pH is readily calculated by a person skilled in the art.

### The Active Ingredient

The active ingredient in the present invention is nicotine. When used herein without further description “nicotine” refers to 3-(1-methyl-2-pyrrolidinyl)-pyridine, in any form, including synthetic nicotine as well as nicotine extracts from tobacco plants, or parts thereof, such as the genus Nicotiana alone or in combination. The nicotine may be formulated in different forms, e.g., in different complexes or salts or as free base.

According to the invention, the liquid pharmaceutical formulation product comprises nicotine in any form to provide a fast transmucosal uptake of the nicotine in the oral cavity of a subject so as to obtain a reduction of the urge to smoke and/or use tobacco, and/or a rapid “nicotine kick” and/or a “nicotine head rush”. Thereby may also be achieved a systemic maintenance level of nicotine.

The nicotine should be in a saliva soluble form to facilitate the subsequent uptake of the nicotine from the saliva in the oral cavity into the systemic circulation of the subject.

In preferred embodiments, the nicotine in any form is primarily selected from the group consisting of the free base form of nicotine, a nicotine salt, a nicotine derivative, a nicotine inclusion complex or nicotine in any non-covalent binding; and mixtures thereof.

Still further the inclusion complex may be a cyclodextrin, such as β-cyclodextrin.

Even more further the nicotine salt may be a tartrate, hydrogen tartrate, citrate or malate.

According to the invention, the uptake of the nicotine through any tissue or mucosa in the oral cavity is improved in relation to the uptake obtained by a liquid nicotine-containing pharmaceutical formulation devoid of alkalizing buffering agents or devoid of alkalizing pH-regulating means.

The nicotine may act as a stimulant to e.g., obtain a rapid reduction of the urge to smoke or to use tobacco.

The most preferable embodiment incorporates nicotine as the free base form or as a water-soluble pharmaceutically acceptable salt, or as an inclusion complex, such as a cyclodextrin complex, e.g., β-cyclodextrin. But any other suitable pharmaceutically acceptable form may also be employed.

Numerous nicotine salts are known, and may be used, e.g., the salts presented in Table 1, such as preferably the tartrate, hydrogen tartrate, citrate, malate, and/or hydrochloride.

TABLE 1
Possible acids used for nicotine salt formation

| | Molar ratio* of |
|---|---|
| Acid | Acid:nicotine |
| Formic | 2:1 |
| Acetic | 3:1 |
| Propionic | 3:1 |
| Butyric | 3:1 |
| 2-Methylbutyric | 3:1 |
| 3-Methylbutyric | 3:1 |
| Valeric | 3:1 |
| Lauric | 3:1 |
| Palmitic | 3:1 |
| Tartaric | 2:1 |
| Citric | 2:1 |
| Malic | 2:1 |
| Oxalic | 2:1 |
| Benzoic | 1:1 |
| Gentisic | 1:1 |
| Gallic | 1:1 |
| Phenylacetic | 3:1 |
| Salicylic | 1:1 |
| Phthalic | 1:1 |
| Picric | 2:1 |
| Sulfosalicylic | 1:1 |
| Tannic | 1:5 |
| Pectic | 1:3 |
| Alginic | 1:2 |
| Hydrochloric | 2:1 |
| Chloroplatinic | 1:1 |
| Silicotungstic | 1:1 |
| Pyruvic | 2:1 |
| Glutamic | 1:1 |
| Aspartic | 1:1 |

*recommended upon manufacturing

### Amount of the Nicotine in the Liquid Pharmaceutical Formulation

The nicotine in any form is according to the invention formulated to provide the subject with a dose to achieve an effect. The effect may be to provide a sense of smoking satisfaction without smoking. Another effect of the administered nicotine in any form may be a reduction of the urge to smoke or use tobacco.

The effect may also be a combination of a reduction of said urge and providing a sense of smoking satisfaction without smoking. The amount of the nicotine should be sufficient to provide such an effect in a subject. This amount may, of course, vary from person to person.

According to the invention, embodiments of the liquid pharmaceutical formulation comprise nicotine in such concentrations that the amount of nicotine delivered at each incidence of administration is about 0.05-10 mg calculated as the free base form of nicotine, preferably about 0.25-6 mg and most preferably about 0.5-4 mg.

Release and Uptake of Nicotine

Presently existing pharmaceutical administration forms for oral administration of nicotine normally provide a slow release and a slow uptake of the nicotine compared to smoking. The slow uptake of the nicotine provides a tₘₐₓ, i.e., the time-point where the nicotine has its maximum level measured in the plasma of venous blood after a single dose at about 30-45 minutes after administration.

The time point for reaching a sense of satisfaction or reduction of urge to smoke or use tobacco after administration is individual, but may in existing pharmaceutical forms for administering nicotine generally be reached after approximately 30 minutes when regarded as coinciding with tmax. According to the present invention, such a sense of satisfaction may be reached after a shorter period of time due to a rapid transmucosal uptake in the oral cavity due to the buffering and/or pH regulation and due to the absence of rate-limiting steps, such as tablet or lozenge melting, tablet or lozenge disintegration and dissolution and chewing gum mastication, followed by drug dissolution.

The Liquid Phase

The liquid phase of the present liquid pharmaceutical formulation may comprise water. The liquid phase may also comprise an alcohol, such as ethanol, glycerol, propylene glycol and polyethylene glycol, or mixtures thereof. It may also comprise one or more lipids. Further it may comprise mixtures of the above ingredients.

Other Additives to the Liquid Pharmaceutical Formulation

Other additives may be added optionally to the liquid pharmaceutical formulation.

Optional additives comprise one or more stabilizing additives, such as those selected from the group consisting of antioxidants including vitamin E, i.e., tocopheroles, vitamin C, i.e., ascorbic acid and its salts, sodium pyrosulfite, butylhydroxytoluene, butylated hydroxyanisole; and preservatives including parabenes, benzalkonium chloride, chlorbutanol, benzyl alcohol, beta-phenylethylal alcohol, cetylpyridinium chloride; and chelating agents, such as EDTA; and galates, such as propyl galate.

Further optional additives comprise one or more additives selected from the group consisting of:
enhancers, such as azone;
vitamins, such as vitamins C and E;
minerals, such as fluorides, especially sodium fluoride, sodium monofluoro phosphate and stannous fluoride;
anti-odours, such as zinc and cyclodextrins;
propellants, such as 1,1,2,2-tetrafluoroethane (HFC-134a), optionally being liquefied, and 1,1,1,2,3,3,3-heptafluororpropane (HFC-227), optionally being liquefied;
sweeteners including one or more synthetic sweetening agents and/or natural sugars, such as those selected from the groups consisting of artificial sweeteners e.g., saccharin and its sodium and calcium salts, aspartame, acesulfame and its potassium salt, thaumatin and glycyrrhizin;

polyhydric alcohols such as sorbitol, xylitol, mannitol and glycerol;

monosaccharides including glucose (also called dextrose), fructose (also called laevulose) and galactose;
disaccharides including saccharose (also called sucrose), lactose (also called milk sugar) and maltose (also called malt sugar);
mixtures of sugars including liquid glucose syrup e.g., starch hydrolysates containing a mixture of chiefly dextrose, maltose, dextrins and water, invert sugar syrup e.g., sucrose inverted by invertase containing a mixture of dextrose, laevulose and water, high sugar content syrups such as treacle, honey and malt extract;

and mixtures thereof;

flavoring and/or aromatizing agents, such as those selected from the group consisting of essential oils obtained by distillations, solvent extractions or cold expressions of fresh or dried flowers, buds, leaves, stems, fruit, seeds, peel, bark, or root e.g., oil of peppermint, spearmint, eucalyptus, wintergreen, niaouli, clove, cardamom, cinnamon, bitter almond, coriander, caraway, ginger, juniper, orange, bitter orange, lemon, grapefruit, mandarine, bergamot, thyme, fennel and rosemary;

natural flavors and aroma agents including either diluted solutions of essential oils or concentrates of flavor components with natural origin from e.g., fruits, berries, nuts, spices, mints, tobacco, cocoa, coffee, tea, vanilla, liquorice, caramel, toffee, honey, wine, liquors and brews;

synthetic flavors and aroma agents consisting of mixtures of chemicals comprising hydrocarbons, alcohols, aldehydes, esters, ketones, ethers and oxides blended to match the natural flavor of e.g., fruits, berries, nuts, spices, mints, tobacco, cocoa, coffee, tea, vanilla, liquorice, caramel, toffee, honey, wine, liquors or brews;

and mixtures thereof.

Surface Active Agents

One or more of the compounds of the liquid pharmaceutical formulation may be solubilized in one or more surface active agents and/or emulsifiers, such as nonionic, cationic, anionic or zwitterionic surfactants, including amphiphilic block copolymers, or mixtures thereof.

Specifically one or more of the compounds of the liquid pharmaceutical formulation may be solubilized in one or more surface-active agents selected from

nonionic surface-active agents including poloxamers, e g: poly(oxypropylene)-poly(oxyethylene) block copolymers, polyoxyethylene alkyl ethers, polyoxyethylene castor oil derivatives, polyoxyethylene sorbitan fatty acid esters, mono- and diglycerides and esters thereof, polyoxyethylene stearates, polyglycerolesters of fatty acids (including polyglycerolpolyricinoleic acid (PGPR)), and sorbitan fatty acid esters,
cationic surface-active agents including secondary, quaternary and tertianary ammonium compounds and cationic phospholipids,
anionic surface-active agents including fatty acid salts, lactylates, especially sodium and/or calcium stearoyllactylate, alkyl sulphates, alkyl sulphonates, latanol, and anionic phospholipids, such as phosphatidylserine,

zwitterionic surface-active agents including zwitterionic phospholipids, such as phosphatidylcholine and phosphatidylethanolamine,
or mixtures thereof,
preferably surface-active agents or mixtures thereof being nonionic.

Method for Delivering Nicotine in Any Form to a Subject

According to the invention, a method for delivering nicotine in any form to a subject comprises the steps of:

a) administering to a subject a liquid pharmaceutical formulation product containing nicotine in any form according to the invention into the oral cavity of the subject, and

b) allowing the nicotine in any form in the liquid pharmaceutical formulation to be mixed with the saliva in the oral cavity and absorbed into the blood plasma of the subject essentially by buccal uptake.

One embodiment results in a tₘₐₓ of nicotine in venous blood of the subject after about 3-30 minutes.

One further embodiment results in a tₘₐₓ of nicotine in venous blood of the subject after about 3-20 minutes.

In still one further embodiment, said nicotine in any form is absorbed resulting in a tₘₐₓ of nicotine in venous blood of the subject after about 3-15 minutes.

Method for Obtaining Reduction of the Urge to Smoke or Use Tobacco

A method for obtaining reduction of the urge to smoke or use tobacco-containing material and/or for providing a sense of smoking satisfaction without smoking according to the invention comprises the steps of:

a) replacing at least partly the tobacco containing material with a liquid pharmaceutical formulation according to any of claims 1-22,

b) administering to a subject a liquid pharmaceutical formulation containing nicotine in any form according to any of claims 1-22 into the oral cavity of the subject, and

c) allowing the nicotine in any form in the liquid pharmaceutical formulation to be absorbed by the subject essentially by buccal uptake.

The administration to the oral cavity takes place by spraying, dropping or pipetting, preferably by spraying, most preferably by spraying under the tongue. The administration is intended for the oral cavity, not for e.g., the lungs or the upper respiratory tract.

In one embodiment said nicotine in any form results in a tₘₐₓ of nicotine in venous blood of the subject after about 3-30 minutes.

In one further embodiment said nicotine in any form results in a tₘₐₓ of nicotine in venous blood of the subject after about 3-20 minutes.

In still one further embodiment said nicotine in any form results in a tₘₐₓ of nicotine in venous blood of the subject after about 3-15 minutes.

Even further embodiments of the method for delivering nicotine to a subject may comprise the steps of combining at least one other method for obtaining reduction of the urge to smoke or use of tobacco.

The liquid pharmaceutical formulation may be used for obtaining a quick and/or sustained and/or complete reduction of the urge to smoke or use tobacco and/or for providing a sense of smoking satisfaction without smoking as further discussed below.

The fast relief provides the subject with a sense of rapid smoking satisfaction without smoking.

One embodiment reduces the urge to smoke or use of tobacco by reaching a tₘₐₓ of nicotine in venous blood of the subject after about 3-30 minutes by the use of a liquid pharmaceutical formulation according to the invention.

One further embodiment reduces the urge to smoke or use tobacco by reaching a tₘₐₓ of nicotine in venous blood of the subject after about 3-20 minutes by the use of a liquid pharmaceutical formulation according to the invention.

Still one further embodiment reduces the urge to smoke or use tobacco by reaching a tₘₐₓ of nicotine in venous blood of the subject after about 3-15 minutes by the use of a liquid pharmaceutical formulation according to the invention.

Cessation of the Urge to Smoke or Use Tobacco

For some of the users, it may be a goal to terminate the usage of nicotine completely, due to several reasons e.g., health, economical, social or behavioral. This may be achieved by further decreasing the delivered amount of nicotine in any form gradually over time. In specific embodiments of the invention, the method described above for obtaining craving relief may further comprise the steps of decreasing the amount of nicotine in the liquid pharmaceutical formulation gradually over time, and/or the steps of reducing the incidence of administration of the liquid pharmaceutical formulation gradually over time, and/or the steps of reducing the dosage size of the liquid pharmaceutical formulation gradually over time, so as to achieve a relief of tobacco craving and/or to achieve a sense of smoking satisfaction. This method results in a weaning process gradually over time.

Different types of smokers reach the sense of reduced craving at different plasma levels of nicotine. This may, of course, affect the individual types of programs for administering a liquid pharmaceutical formulation according to the invention. Different types of smokers include e.g., peak seekers or smokers that crave for a plasma level of nicotine constantly being above the level below which withdrawal symptoms occur.

One strategy may be to lower the frequency of administering the liquid pharmaceutical formulation. Other embodiments include varying the dose of the nicotine in said liquid pharmaceutical formulation as well as the combination of these two embodiments.

Systems for Delivering Nicotine and for Obtaining Craving Relief

According to the invention there is a system for delivering nicotine in any form to a subject. Such a system comprises a liquid pharmaceutical formulation according to the invention and at least one other means for obtaining reduction of the urge to smoke.

Another system according to the invention may be a system for obtaining reduction of the urge to smoke or use tobacco and/or for providing a sense of smoking satisfaction without smoking. Such a system comprises a liquid pharmaceutical formulation according to the invention and at least one other method for obtaining reduction of the urge to smoke or use tobacco. Other methods may be a concomitant or concurrent method selected from the group consisting of administration through chewing gums, nasal sprays, transdermal patches, inhaling devices, lozenges, tablets and parenteral methods, subcutaneous methods, intravenous methods, rectal methods, vaginal methods and transmucosal methods; or use of tobacco.

In a specific embodiment, the at least one other method comprises administration of nicotine.

Use of the Liquid Pharmaceutical Formulation

The use of the liquid pharmaceutical formulation according to the invention is for obtaining a fast and/or complete reduction of the urge to smoke and use tobacco or for providing a sense of smoking without smoking as described above.

The dose of the nicotine is chosen to give the subject an individual sensory perception and satisfaction with an effect of the nicotine in any form. The use of the liquid pharmaceutical formulation may also be a sole use according to the invention or a combination with other means or methods known in the field of drug abuse. Specifically, the present invention may be used in combination with other means as described above in the methods in the paragraphs above.

The use may give a quick reduction of the urge to smoke or use tobacco whereby is reached a tₘₐₓ of nicotine in venous blood after about 3-20 minutes.

In a specific embodiment, the use of the liquid pharmaceutical formulation according to the invention will reduce the urge to smoke or use tobacco by reaching a tₘₐₓ of nicotine in venous blood of the subject after about 3-15 minutes.

According to the invention, a use of a liquid pharmaceutical formulation according to the invention is for delivering nicotine in any form to a subject.

In one embodiment, the delivering of nicotine in any form results in a tₘₐₓ of nicotine in venous blood of the subject after about 3-30 minutes.

In another embodiment, the delivering of nicotine in any form results in a tₘₐₓ of nicotine in venous blood of the subject after about 3-20 minutes.

In still another embodiment, the delivering of nicotine in any form results in a tₘₐₓ of nicotine in venous blood of the subject after about 3-15 minutes.

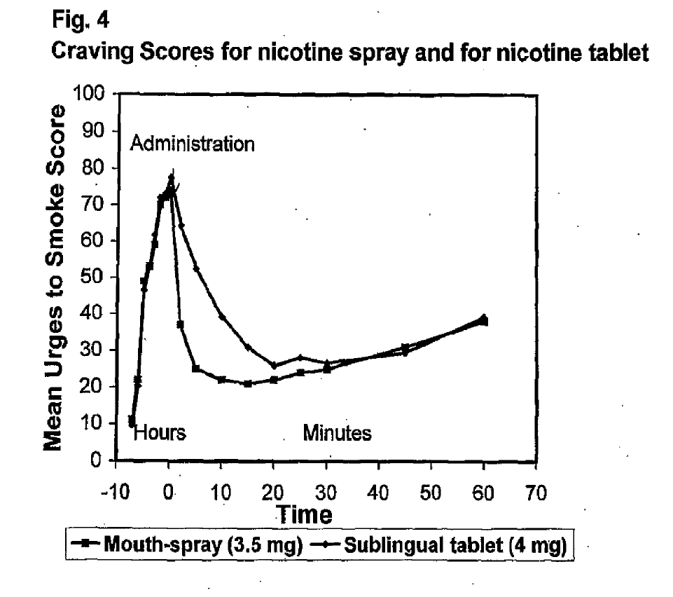

As readily shown and concluded from the figures, e.g., FIG. 4, the shorter the tₘₐₓ the faster the relief of the craving, i.e., of the urge to smoke.

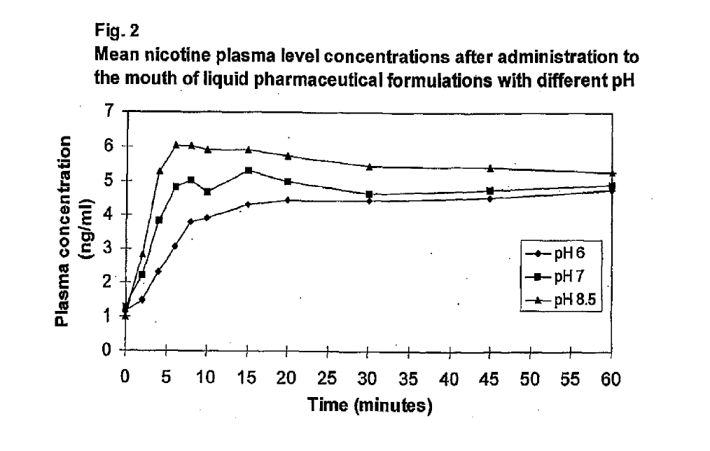

All publications, patents, and patent applications cited herein are hereby incorporated by reference in their entirety for all purposes. The examples set forth below are non-limiting and for illustrating the present invention. Alternatives and variations of the below examples within the scope of the present invention as per the below claims may be carried out by a person skilled in the art. Ingredients as per the below examples may be exchanged for equivalent ingredients, preferably as per above. The formulations according to Examples 2 and 3 were made for comparative purposes as seen from FIG. 2.

### EXAMPLE 1

Manufacturing of a 1000 ml formulation with 10 mg nicotine/ml and around pH 8.5.

Mixture 1

To a beaker containing 800 ml water of 90° C. was added 0.7 g methyl para-hydroxybenzoate, acting as preservative, and 0.3 g propyl para-hydroxybenzoate, acting as preservative. The additives were dissolved during stirring for about 10 minutes. Then was added 10.45 g sodium dihydrogen phosphate, acting as buffering agent, and 0.5 g EDTA, acting as chelating agent, to the solution, which was stirred for about 5 minutes. Then the solution was cooled to 30° C. during stirring.

Mixture 2

To a beaker containing 15.9 g ethanol of room temperature, acting as solvent, was added 0.045 g peppermint oil, acting as flavoring agent. The liquid was mixed for 2 minutes.

Final Mixture

Mixture 2 was added during stirring to a beaker containing 150 ml water. Gently 10 g nicotine (base) was added to the beaker. Then Mixture 1 was added to the beaker and stirred for 5 minutes. The pH of the Final mixture was checked and adjusted to about pH 8.5 with sodium hydroxide (20%) and to volume with water.

### EXAMPLE 2

Manufacturing of a 1000 ml formulation with 10 mg nicotine/ml and around pH 7.0.

This Example 2 differs from Example 1 only for pH. The formulation according to Example 2 contains a non-alkalizing buffering agent. This formulation was for use as a comparison in FIG. 2.

Mixture 1

To a beaker containing 800 ml water of 90° C. was added 0.7 g methyl para-hydroxybenzoate, acting as preservative, and 0.3 g propyl para-hydroxybenzoate, acting as preservative. The additives were dissolved during stirring for about 10 minutes. Then was added 10.45 g sodium dihydrogen phosphate, acting as buffering agent, and 0.5 g EDTA, acting as chelating agent, to the solution, which was stirred for about 5 minutes. Then the solution was cooled to 30° C. during stirring.

Mixture 2

To a beaker containing 15.9 g ethanol of room temperature, acting as solvent, was added 0.045 g peppermint oil, acting as flavoring agent. The liquid was mixed for 2 minutes.

Final Mixture

Mixture 2 was added during stirring to a beaker containing 150 ml water. Gently 10 g nicotine (base) was added to the beaker. Then Mixture 1 was added to the beaker and stirred for 5 minutes. The pH of the Final mixture was checked and adjusted to about pH 7.0 with hydrochloric acid and to volume with water.

### EXAMPLE 3

Manufacturing of a 1000 ml formulation with 10 mg nicotine/ml and around pH 6.0.

This Example 3 differs from Example 1 only for pH. The formulation according to Example 3 contains a non-alkalizing buffering agent. This formulation was for use as a comparison in FIG. 2.

Mixture 1

To a beaker containing 800 ml water of 90° C. was added 0.7 g methyl para-hydroxybenzoate, acting as preservative, and 0.3 g propyl para-hydroxybenzoate, acting as preservative. The additives were dissolved during stirring for about 10 minutes. Then was added 10.45 g sodium dihydrogen phosphate, acting as buffering agent, and 0.5 g EDTA, acting as chelating agent, to the solution, which was stirred for about 5 minutes. Then the solution was cooled to 30° C. during stirring.

Mixture 2

To a beaker containing 15.9 g ethanol of room temperature, acting as solvent, was added 0.045 g peppermint oil, acting as flavoring agent. The liquid was mixed for 2 minutes.

Final Mixture

Mixture 2 was added during stirring to a beaker containing 150 ml water. Gently 10 g nicotine (base) was added to the beaker. Then Mixture 1 was added to the beaker and stirred for 5 minutes. The pH of the Final mixture was checked and adjusted to about pH 6.0 with hydrochloric acid and to volume with water.

### EXAMPLE 4

Manufacturing of a 1000 ml formulation with 17.5 mg nicotine/ml and around pH 9.0.

Mixture 1

To a beaker containing 600 ml water of room temperature was added 12.0 g Synperonic® PE/F27, being a poloxamer acting as non-ionic surface active agent. The additive was dissolved during stirring for about 20 minutes. Then was added 0.5 g EDTA, acting as chelating agent, and 0.4 g sodium saccharin, acting as sweetener, to the liquid which was stirred until all ingredients were dissolved. Then was added 16.8 g sodium hydrogen carbonate, acting as buffering agent, and the solution was stirred until a clear solution was obtained.

Mixture 2

To a beaker containing 250.0 g ethanol of room temperature, acting as solvent, was added 0.7 g methyl para-hydroxybenzoate acting as preservative, and 0.3 g propyl para-hydroxybenzoate acting as preservative. The liquid was mixed until the ingredients were dissolved. Then was added 5.0 g peppermint oil, acting as flavoring agent, and 1.5 g aroma agent. The liquid was mixed until a clear solution was obtained.

Final Mixture

Mixture 2 was gently added to Mixture 1 during stirring for about 1 minute. Then was added 17.5 g nicotine (base) and the liquid was stirred for about 2 minutes. The pH of the Final mixture was checked and adjusted to around pH 9.0 with hydrochloric acid. The Final mixture was transferred to a 1000 ml volumetric flask and adjusted to 1000 ml volume by water. Finally the pH of the solution was checked to remain at around pH 9.0.

### EXAMPLE 5

Manufacturing of a 1000 ml formulation with 14.3 mg nicotine/ml and around pH 9.0.

Mixture 1

To a beaker containing 600 ml water of room temperature was added 20.0 g Synperonic® PE/F27 being a poloxamer, acting as non-ionic surface active agent. The additive was dissolved during stirring for about 20 minutes. Then was added 2.0 g Acesulfame K, acting as sweetener, to the liquid which was stirred until all ingredients were dissolved. Then was added 20.0 g sodium hydrogen carbonate, acting as buffering agent, and the liquid was stirred until a clear solution was obtained.

Mixture 2

To a beaker containing 95.0 g ethanol of room temperature, acting as solvent, was added 3.5 g peppermint oil, acting as flavoring agent, and 1.0 g aroma agent. The liquid was mixed until a clear solution was obtained.

Final Mixture

Mixture 2 was gently added to Mixture 1 during stirring for about 1 minute. Then was added 14.3 g nicotine (base) and the liquid was stirred for about 2 minutes. The pH of the Final mixture was checked and adjusted to around pH 9.0 with hydrochloric acid. The Final mixture was transferred to a 1000 ml volumetric flask and adjusted to 1000 ml volume by water. Finally the pH of the solution was checked to remain at around pH 9.0.

The formulation according to Example 5 is a preferred composition.

### EXAMPLE 6

Manufacturing of a 1000 ml formulation with 14.3 mg nicotine/ml and around pH 9.0.

Mixture 1

To a beaker containing 600 ml water of room temperature was added 20.0 g Synperonic® PE/F27 being a poloxamer, acting as non-ionic surface active agent. The additive was dissolved during stirring for about 20 minutes. Then was added 0.2 g benzalkonium chloride, acting as preservative, and 2.0 g Acesulfame K, acting as sweetener, to the liquid which was stirred until all ingredients were dissolved. Then was added 20.0 g sodium hydrogen carbonate, acting as buffering agent, and the liquid was stirred until a clear solution was obtained.

Mixture 2

To a beaker containing 95.0 g ethanol of room temperature, acting as solvent, was added 3.5 g peppermint oil, acting as flavoring agent, and 1.0 g aroma agent. The liquid was mixed until a clear solution was obtained.

Final Mixture

Mixture 2 was gently added to Mixture 1 during stirring for about 1 minute. Then was added 14.3 g nicotine (base) and the liquid was stirred for about 2 minutes. The pH of the Final mixture was checked and adjusted to around pH 9.0 with hydrochloric acid. The Final mixture was transferred to a 1000 ml volumetric flask and adjusted to 1000 ml volume by water. Finally the pH of the solution was checked to remain at around pH 9.0.

The formulation according to Example 6 is a another preferred composition.

### EXAMPLE 7

Manufacturing of a 1000 ml formulation with 14.3 mg nicotine/ml and around pH 9.0.

Mixture 1

To a beaker containing 600 ml water of room temperature was added 20.0 g Synperonic® PE/F27 being a poloxamer, acting as non-ionic surface active agent. The additive was dissolved during stirring for about 20 minutes. Then was added 0.5 g EDTA, acting as chelating agent, and 2.0 g Acesulfame K, acting as sweetener, to the liquid which was stirred until all ingredients were dissolved. Then was added 20.0 g sodium hydrogen carbonate, acting as buffering agent, and the liquid was stirred until a clear solution was obtained.

Mixture 2

To a beaker containing 95.0 g ethanol of room temperature, acting as solvent, was added 0.7 g methyl para-hydroxybenzoate acting as preservative, and 0.3 g propyl para-hydroxybenzoate acting as preservative. The liquid was mixed until the ingredients were dissolved. Then was added 3.5 g peppermint oil, acting as flavoring agent, and 1.0 g aroma agent. The liquid was mixed until a clear solution was obtained.

Final Mixture

Mixture 2 was gently added to Mixture 1 during stirring for about 1 minute.

Then was added about 2 ml sodium hydroxide (50%) and 4 g nicotine bitartrate. The pH of the Final mixture was not allowed to decrease below pH 8 during the addition of the nicotine bitartrate. The preceding procedure with adding of sodium hydroxide and nicotine bitartrate was repeated until totally 40.7 g nicotine bitartrate was added. The pH of the Final mixture was adjusted to around pH 9.0. The Final mixture was transferred to a 1000 ml volumetric flask and adjusted to 1000 ml volume by addition of water. Finally the pH of the solution was checked to remain at around pH 9.0.

### EXAMPLE 8

Manufacturing of a 1000 ml formulation with 17.5 mg nicotine/ml and pH 10.94.

To a beaker containing 950 ml water of room temperature was added 17.5 g nicotine (base) during stirring for about 5 minutes. The volume was adjusted to 1000 ml volume by addition of water. Finally the pH was checked.

### EXAMPLE 9

Manufacturing of a 1000 ml formulation with 17.5 mg nicotine/ml and pH 11.55.

To a beaker containing 950 ml water of room temperature was added 35 g sodium carbonate anhydrous during stirring until complete dissolution. Then 17.5 g nicotine (base) was added during stirring for about 5 minutes. The volume was adjusted to 1000 ml volume by addition of water. Finally the pH was checked.

### EXAMPLE 10

Manufacturing of a 1000 ml formulation with 15.65 mg nicotine/ml and pH 11.79.

To a beaker containing 950 ml water of room temperature was added 158 g glycine sodium salt during stirring until complete dissolution. Then 15.65 g nicotine (base) was added during stirring for about 5 minutes. The volume was adjusted to 1000 ml volume by addition of water. Finally the pH was checked.

### EXAMPLE 11

Buffer Capacity Determinations

Method: 10.0 ml of the respective below solutions was titrated with 0.1 M HCl to pH 7.0. The amount of 0.1 M HCl needed to decrease pH from 9.0 to 8.0 was determined

Definitions: (1) Sodium hydrogen carbonate (NaHCO3). Mw: 84.0
(2) Disodium phosphate dodecahydrate (Na2HPO4, 12H2O) Mw: 358.1

| | Batch | | | | | |
|---|---|---|---|---|---|---|
| Ingredient | DKN0293 | DKN0294 | DKN0295 | DKN0296 | DKN0290 | DKN0291 |
| Nicotine (mg/ml) | | | 10.0 | | 10.0 | 10.0 |
| NaHCO₃ (mg/ml) | 16.8 | | 16.8 | 8.4 | | |
| Na₂HPO₄, 12H₂O | | 71.6 | | 35.8 | 71.6 | |
| (mg/ml) | | | | | | |
| Purified water ad | 1 ml | 1 ml | 1 ml | 1 ml | 1 ml | 1 ml |
| Buffer Capacity | 26.5 | 9.5 | 50 | 15.8 | 40 | 29 |
| PH = 9.0-8.0 | | | | | | |
| (mekv/l) | | | | | | |

All solutions were adjusted to a pH of 9.0 when needed. A higher pH may cause irritation and corrosion, which might be harmful to the tissue of the oral cavity.
16.8 mg/ml of NaHCO3 corresponds to 0.2M.
71.6 mg/ml of Na2HPO4, 12H2O corresponds to 0.2M.
8.4 mg/ml of NaHCO3 corresponds to 0.1M.
35.8 mg/ml of Na2HPO4, 12H2O corresponds to 0.1M

Nicotine base has an alkalizing effect, but has too weak a buffering capacity on its own. The buffering capacity of the formulation is significantly and sufficiently increased when a buffering agent is added.

The above data clearly show that the present formulations have a good buffering capacity, providing for the desired rapid transmucousal uptake of nicotine.

A liquid pharmaceutical formulation according to the present invention may be administered using suitable devices being available on the market, e.g., spray devices.

### Analysis of Nicotine

The analysis of nicotine uptake and of the effect of the invention may be done according to standard procedures known in the art, e.g., using a bioanalysis for the determination of nicotine in the plasma of a subject.

### Effects of the Invention

Comparative tests were conducted as described above under Legend of figures.

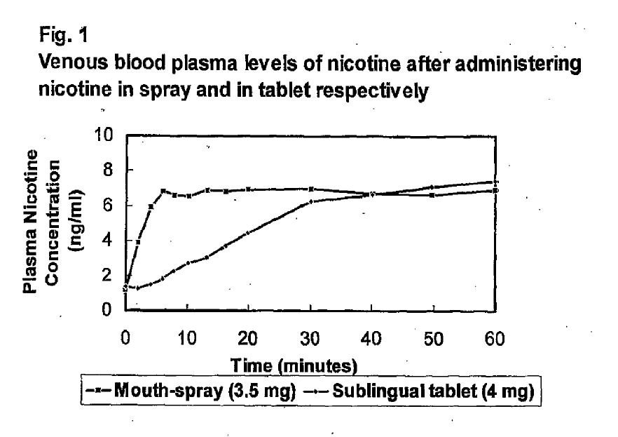

FIG. 1 shows that with a liquid pharmaceutical formulation according to the present invention the venous blood plasma level of nicotine ascends significantly more rapidly than with Nicorette Microtab®. Nicorette Microtab® has the same pharmacokinetic profile as, i.e., is pharmacologically equivalent with, Nicorette Gum® and all other nicotine chewing gums currently on the market. Nicotine chewing gums presently represent around half of the world sales of medicinal nicotine-containing products for smoking cessation and similar indications.

FIG. 2 shows that the higher the pH of a liquid pharmaceutical formulation according to the present invention the faster the absorption kinetics and the higher the plasma concentration of nicotine.

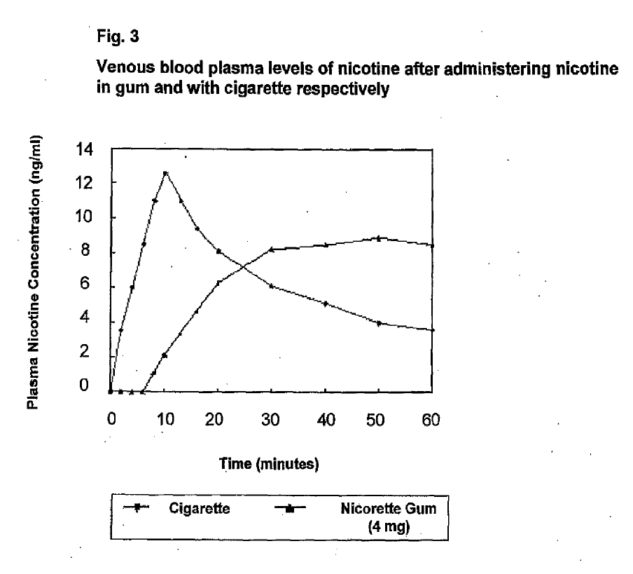

FIGS. 3 and 4 further show that a formulation according to the present invention provides for a fast craving relief manifested through a significantly faster reduction in the urge to smoke compared to known oral nicotine formulations.

Use for Therapy, Treatment and Manufacturing

The liquid pharmaceutical formulation product according to the invention may be used in therapy. Said therapy may be a treatment of a disease or medical indication selected from the group consisting of reduction in use of tobacco, cessation of use of tobacco, other use of tobacco, temporary abstinence from abstaining from use of tobacco, Alzheimer's disease, Crohn's disease, Parkinson's disease, Tourette's syndrome, and ulcerative colitis; and weight control.

Nicotine in any form may be used for the manufacturing of a liquid pharmaceutical formulation according to the invention for the treatment of a disease or medical indication selected from the group consisting of reduction in use of tobacco, cessation of use of tobacco, other use of tobacco, temporary abstinence from abstaining from using tobacco, Alzheimer's disease, Crohn's disease, Parkinson's disease, Tourette's syndrome, and ulcerative colitis; and weight control.

What is claimed:

1. A liquid pharmaceutical formulation for use in the treatment of addiction to tobacco or nicotine, comprising:
(i) nicotine in free nicotine base form; and
(ii) a buffering agent,
(iii) the liquid pharmaceutical formulation being adapted for administration to the oral cavity of a subject by spraying, dropping or pipetting, and
(iv) the liquid pharmaceutical formulation being adapted so as to achieve a tₘₐₓ of nicotine in venous blood of the subject within from about 3 to about 30 minutes after an incidence of administration of the liquid pharmaceutical formulation.

2. The liquid pharmaceutical formulation of claim 1, wherein the buffering agent comprises a carbonate, a glycinate, a phosphate, a glycerophosphate, an acetate, a gluconate, or citrate of an alkali metal, or any combination thereof.

3. The liquid pharmaceutical composition of claim 1, further comprising a pH regulating agent.

4. The liquid pharmaceutical composition of claim 3, wherein the pH regulating agent comprises sodium hydroxide, potassium hydroxide, calcium hydroxide, or calcium oxide.

5. The liquid pharmaceutical formulation of claim 1, wherein the liquid phase comprises an alcohol.

6. The liquid pharmaceutical formulation of claim 1, wherein the liquid phase comprises one or more lipids.

7. The liquid pharmaceutical formulation of claim 1, wherein the liquid pharmaceutical formulation comprises water, an alcohol, a lipid, or any combination thereof.

8. The liquid pharmaceutical formulation of claim 1, further comprising a flavor agent.

9. The liquid pharmaceutical formulation of claim 1, further comprising an antioxidant, a preservative, a chelating agent, or any combination thereof.

10. The liquid pharmaceutical formulation of claim 1, further comprising a thickening agent, an enhancer, a vitamin, a mineral, an anti-odorant, a propellant, a sweetener, or any combination thereof.

11. The liquid pharmaceutical formulation of claim 1, further comprising a surfactant.

12. The liquid pharmaceutical formulation of claim 1, further comprising a sodium hydrogen carbonate acting as buffering agent, water acting as a solvent, ethanol acting as co-solvent, a poloxamer acting as surface active agent, EDTA acting as chelating agent, Acesulfame K acting as sweetener, optionally one or more preservatives, optionally one or more flavoring or aroma agents, or any combination thereof.

13. The liquid pharmaceutical formulation of claim 1, wherein the formulation provides an essentially complete reduction of the urge to smoke or use tobacco without smoking.

14. The liquid pharmaceutical formulation according to claim 1, wherein the formulation is adapted to comprise nicotine in such concentration that the amount of nicotine delivered at an incidence of administration is 0.05-10 mg.

15. The liquid pharmaceutical formulation of claim 14, wherein the amount of nicotine delivered at an incidence of administration is 0.25-6 mg.

16. The liquid pharmaceutical formulation of claim 16, wherein the amount of nicotine delivered at an incidence of administration is 0.5-4 mg.

17. The liquid pharmaceutical formulation of claim 1, whereby the liquid pharmaceutical formulation is adapted such that the ₜₘₐₓ of nicotine in venous blood is reached from about 3 to about 20 minutes after an incidence of administration of the formulation.

18. The liquid pharmaceutical formulation of claim 17, wherein the liquid pharmaceutical formulation is adapted such that the tₘₐₓ of nicotine in venous blood is reached from 3 to 15 minutes after an incidence of administration of the formulation.

19. A system for delivering nicotine to a subject, the system comprising a liquid pharmaceutical formulation that comprises:
(i) nicotine in free nicotine base form; and
(ii) a buffering agent; and
(iii) the formulation being adapted for administration to the oral cavity of a subject by spraying, dropping or pipetting, and
(iv) the formulation being adapted so as to achieve a tₘₐₓ of nicotine in venous blood of the subject within from about 3 to about 30 minutes after an incidence of administration of the formulation; and
the system further comprising at least one other method of nicotine administration selected from the group consisting of chewing gums, nasal sprays, transdermal patches, inhaling devices, lozenges, tablets and parenteral methods, subcutaneous methods, intravenous methods, rectal methods, vaginal methods, or transmucousal methods.
